(19) United States
(12) Patent Application Publication
(10) Pub. No.: US 2026/0363880 A1
(43) Pub. Date: Nov. 19, 2026

(54) PROSTHETIC OR ORTHOTIC KNEE JOINT AND METHOD FOR THE CONTROL THEREOF

(71) Applicant: OTTO BOCK HEALTHCARE PRODUCTS GMBH, Wien (AT)

(72) Inventors: Roland PAWLIK, Wien (AT); Ute HANTSCH, Wien (AT)

(21) Appl. No.: 19/135,677

(22) PCT Filed: Dec. 5, 2023
(86) PCT No.: PCT/EP2023/084352

§ 371 Date: Sep. 5, 2025

(30) Foreign Application Priority Data
Dec. 6, 2022 (DE) 10 2022 132 341.2

(51) Int. Cl. A61F 2/64; A61F 2/50; A61F 2/68; A61F 2/70; A61F 2/74; A61F 5/01

(52) U.S. Cl. CPC A61F 2/64; A61F 2/70; A61F 2/748; A61F 5/0125; A61F 2002/5004; A61F 2002/5006; A61F 2002/503; A61F 2002/5033; A61F 2002/6854; A61F 2002/701; A61F 2005/0144; A61F 2005/0158; A61F 2005/0167; A61F 2005/0169

(57) ABSTRACT

The invention relates to a prosthetic or orthotic knee joint comprising an upper part (10) and a lower part (20) mounted on the upper part (10) such that it can pivot about a pivot axis (15), and a resistance unit (30) between the upper part (10) and the lower part (20), with which the pivotability of the upper part (10) relative to the lower part (20) can be influenced at least in the flexion direction, comprising a control unit (40) which is coupled to the resistance unit (30) and via which the resistance against a flexion is changed according to the position of the upper part (10) relative to the lower part (20), wherein the control unit (40) is designed to block or significantly inhibit a flexion movement at a blocking angle (a) when reaching or before reaching a mechanical end stop (80).

### CROSS REFERENCE TO RELATED APPLICATIONS

The present application is the U.S. National Phase under 35 U.S.C. § 371 of International Application No. PCT/EP2023/084352, filed on 5 Dec. 2023, which claims priority from German Patent Application No. 10 2022 132 341.2, filed on 6 Dec. 2022, the contents of which are hereby incorporated by reference in their entirety.

The invention relates to a prosthetic knee joint or orthotic knee joint having an upper part and a lower part, which is mounted pivotably about a pivot axis on the upper part, and having a resistance device between the upper part and the lower part, with which the pivotability of the upper part relative to the lower part at least in flexion direction can be influenced, and having a control device, which is coupled to the resistance device and by which the resistance against a flexion is varied as a function of the setting of the upper part with respect to the lower part. The invention likewise relates to a method for controlling such a prosthetic knee joint or orthotic knee joint.

Prosthetic knee joints replace knee joints of a human body that are no longer present or not present, and have an upper part and a lower part that are mounted displaceably with respect to one another about a pivot axis. The pivot axis may be configured to be stationary or nonstationary. The prosthetic knee joint in a simple construction has a single spatially defined pivot axis and is configured in the form of a hinge joint, a resistance device being arranged between the upper part and the lower part, for example a linearly acting hydraulic damper, in order to influence the pivoting movement about the pivot axis. The prosthetic knee joint may comprise a locking device with which the prosthetic knee joint is locked in the desired setting, particularly in an extended setting, so that a blocking knee joint is formed.

Orthoses stabilize or guide the movement of the natural knee joint and can limit the pivoting angle, for example in order to prevent full extension or full flexion of the natural knee for medical reasons. Orthotic knee joints are arranged in orthoses that cross the natural knee joint when worn. The orthotic knee joint also comprises an upper part and a lower part, on which splints or other components by which the orthotic knee joint is fixed to the thigh or the shank are arranged. In general, knee orthoses comprise two orthotic knee joints, one of which is arranged medially and the other laterally with respect to the natural knee joint. In order to influence the pivoting movement of the thigh with respect to the shank, resistance devices or dampers are arranged in or on the orthosis and act between the upper part and the lower part of the orthosis.

In order to assist the movement, active drives may be arranged on an orthosis as well as on a prosthesis, these drives being configured for example as electric motors or force accumulators in order to assist a flexion or extension in the situation in question. The force accumulators or drives may also be configured to oppose the respective movement and be arranged on the orthosis or prosthesis. The influencing of the movement of the upper part relative to the lower part with the resistance device, or the drive, may be adapted to the situation or load in question. This is done, for example, by a control device which, on the basis of sensor values, activates, deactivates or modulates an actuator that is assigned to the resistance device or is part of the resistance device, and varies parameters of the resistance device in order to provide the respectively desired resistance. The actuator may, for example, be a relay by which a switching valve can be switched between two states. An electric motor may bring a control valve into the desired setting in order to generate the required resistance, for example in a flow channel. An electromagnet may generate the desired magnetic field in order to vary a magnetorheological fluid in respect of its viscosity so as to generate the desired resistance against a displacement. The control device comprises a microprocessor or a data processing device, with which the sensor data are processed promptly, particularly in real time, and used as a basis for the control. With the resistance device, it is in principle also possible to fully suppress the pivoting or pivotability of the upper part with respect to the lower part.

Because of the design construction, prosthetic knee joints and orthotic knee joints have a predefined maximum pivoting angle both in the extension direction and in the flexion direction. At the end of the maximum pivotability, there are mechanical end-stops which prevent a further movement of the upper part and lower part in the extension direction or flexion direction. Particularly in the event of rapid movements, the problem may arise that the prosthesis components are brought unbraked or with a high speed into the mechanical end-stop, which leads to a large impulse in the prosthetic knee joint, or orthotic knee joint. This is uncomfortable for the user of the artificial knee joint and may also cause damage to the orthosis or prosthesis, particularly to the mechanical components of the prosthetic knee joint and orthotic knee joint. In order to prevent a hard stop of the lower part into the mechanical extension stop, or flexion stop, mechanical dampers are provided, for example elastomer elements, which because of the solid-state damping become effective and convert the kinetic energy shortly before the mechanical end-stop is reached.

Prostheses and orthoses are intended to make it easier for the user to perform everyday movements. In the case of prosthetic knee joints and orthotic knee joints, the main uses are sitting, standing as well as walking and the transitions between them. There are furthermore a multitude of activities that differ from these states or intended purposes, for example daily chores in the household, picking up things that have fallen down, shopping or the like. Engaging with pets or infants also requires movements that may be challenging for users of prosthetic knee joints and orthotic knee joints. Instead of bending forward with the upper body, users of prostheses or orthoses often have to bend at the knee. Since this is comparatively strenuous, it is necessary to carry out compensating movements or refrain from such activities, which restricts quality of life.

It is an object of the present invention to provide a prosthetic knee joint or orthotic knee joint and a method for controlling such a knee joint, with which comfort can be increased and additional areas of use can be achieved for the user.

This object is achieved by a prosthetic knee joint or orthotic knee joint having an upper part and a lower part, which is mounted pivotably about a pivot axis on the upper part, and having a resistance device between the upper part and the lower part, with which the pivotability of the upper part relative to the lower part at least in flexion direction can be influenced, and having a control device, which is coupled to the resistance device and by which the resistance against a flexion is varied as a function of the setting of the upper part and the lower part, wherein the control device is adapted to block or more strongly damp a flexion movement at a blocking angle when or before a mechanical end-stop is reached. The sensor-dependent activation, deactivation or modulation of the resistance device for the blocking or increased damping of a flexion movement when a blocking angle is reached, before the mechanical end-stop is reached, either prevents the mechanical end-stop from being reached or damps the reaching of the mechanical end-stop so greatly that the prosthetic knee joint or orthotic knee joint is not damaged, or the mechanical loads are reduced. An increased flexion resistance is provided even before the mechanical end-stop is reached, although it may also be proportioned so that the mechanical end-stop is still reached, albeit with a reduced speed, in particular a greatly reduced speed.

At the blocking angle, which preferably lies before the mechanical end-stop, the resistance against a flexion due to the resistance device is maximal, and in particular the flexion movement is blocked at the blocking angle before the mechanical end-stop is reached.

The control device is in one embodiment adapted to increase the flexion resistance over a defined angle range to the extent of blocking before the blocking angle is reached. In the control device, the blocking angle is ascertained or calculated or the defined blocking angle is monitored. If the joint angle of the upper part relative to the lower part exceeds a threshold value for the angle range, for example if the lower part approaches a flexion angle closer than 15° to the blocking angle, the resistance is increased even before the blocking angle is reached in order to increase a smooth reduction of the flexion speed by an increase of the flexion resistance over an angle range. The more closely the lower part approaches the blocking angle, the greater the flexion resistance due to the resistance device advantageously becomes.

The control device is in one embodiment coupled to at least one sensor for recording the joint angle between the upper part and the lower part or for recording the orientation of the upper part in space, and furthermore adapted to vary the resistance due to the resistance device on the basis of the sensor values of the sensor or sensors. This variation takes place in particular after a threshold value of the joint angle is reached. Complementarily or alternatively, the variation of the resistance takes place on the basis of the duration of an unchanged joint angle, a defined orientation of the upper part in space and/or a blocking command. The effect of the variation is, in particular, that the resistance is increased to the extent of blocking against a flexion. In this way, the resistance may be maximized and a further flexion movement suppressed in an arbitrary angular setting because of a blocking command. Locking may likewise take place in the event of a static state over a certain period of time, i.e. in the event of an unchanged setting of the upper part with respect to the lower part. The resistance is also increased, optionally to the extent of blocking, in the event of a predetermined orientation or in the event that a predetermined joint angle is reached. For example, the user may therefore carry out squatting or kneeling activities more easily since the knee joint that is blocked in the flexion direction can be loaded in the flexion direction by the user and the blocking generates a support, or counter-bearing, so that it is no longer necessary for the musculature to apply a moment that extends the knee. In addition, support by an active drive and/or the musculature of the healthy extremities is also possible when standing up again.

In order to achieve release of the blocked knee joint, the control device is coupled to at least one sensor for recording a release signal, with which the blocking is negated. The release signal may be a manual signal via a keypad, a switch or the like. Alternatively, a particular movement, a particular force profile, a state change or an orientation or a change of an orientation, for example of the upper part, may lead to release of the prosthetic knee joint or orthotic knee joint. If the upper part moves through a particular pivoting angle in the extension direction relative to the lower part, for example, this may be interpreted as a signal to bring about release of the resistance device against the flexion movement.

In one embodiment, the control device is adapted to increase the flexion resistance in the event of increasing flexion. The increase of the resistance is coupled positively with the variation of the joint angle, i.e. the more the joint angle approaches the blocking angle, the greater the resistance becomes. The coupling may take place linearly, progressively or degressively.

Particularly in the case of a pneumatic or hydraulic embodiment of the resistance device, at least one control valve or an adjustment device is arranged in the resistance device and is coupled to an actuator that is activated, deactivated or modulated by the control device. Prosthetic knee joints or orthotic knee joints often already contain hydraulic or pneumatic damper devices in order to influence the most frequent or most important movements. By a corresponding control device, the control valve or an adjustment device may be activated by an actuator in such a way that the desired resistance is set, in particular the flexion resistance is increased, before the mechanical end-stop is reached, and in particular blocking of the joint in the flexion direction is brought about before the mechanical end-stop is reached. The resistance device may be configured as a linear damper or rotary damper, in both of which embodiments the resistance device may be based on a hydraulic or pneumatic principle. Alternatively or complementarily, a magnetorheological resistance device may be arranged in the hydraulic resistance device or assigned thereto. With a magnetorheological fluid, the hydraulic resistance device may be operated and the viscosity may additionally be varied in the desired way by a controllable electromagnet. In principle, it is also possible to use an active drive as a resistance device.

According to one embodiment of the control device, parameters that lead to the blocking or the increase of the flexion resistance are stored in it, and the parameters for the blocking can be set in the control device. It is therefore possible to define resistance curves, signal durations for blocking activations, release signals and positions of the blocking angles that are adapted to the patient.

According to one embodiment, the blocking angle at which a further flexion is blocked or maximum damping is provided by the resistance device is set at between 2° and 40°, in particular between 5° and 30° before the mechanical end-stop. This ensures that, in the event of permanent loading of a hydraulic resistance device or of a pneumatic resistance device, the possibly existing leakage or delays in the action of the actuator do not lead to a hard stop taking place in the mechanical end-stop.

In the method for controlling such a prosthetic knee joint or orthotic knee joint, the flexion resistance is increased before the blocking angle is reached, after reaching a threshold value of the joint angle, in the event of increasing flexion and/or in the event of a constant joint angle and/or constant orientation of the upper part in space over a defined period of time and/or in the event of a blocking signal.

The resistance is in one embodiment of the method increased linearly or progressively with an increasing approach to the blocking angle. The flexion resistance is in one embodiment increased over an angle range of more than 5° before the blocking angle is reached, in order to have a sufficient period of time or a sufficient distance to increase the damping and reduce the flexion speed.

In one embodiment of the method, the control device is coupled to a foot part that is fitted to a tibial tube in the case of a prosthetic knee joint, or a tibial splint in the case of an orthotic knee joint. Release or blocking of the prosthetic knee joint or orthotic knee joint takes place on the basis of the setting and/or a load of the foot part. Blocking or release of the prosthetic knee joint or orthotic knee joint is carried out depending on the way in which the foot part is loaded, whether there is a forefoot load, whether flexion is taking place or whether there is bending of a sole part or of a sole element. For this purpose, sensors that are coupled to the control device may be arranged on the lower part or the foot part, and the blocking or release, or increase of the resistance or reduction of the flexion resistance, is carried out on the basis of sensor data.

According to one development of the method, the position of the center of mass of the body relative to the treated leg is recorded or ascertained, and a decision relating to an increase of the resistance or blocking is made during a movement on the basis of the profile of the center of mass of the body. The center of mass of the body is, for example, ascertained or calculated relative to the base point or the force transmission point of the ground reaction force, the pivot axis of the ankle joint or for example of the knee joint. With the aid of the profile of the center of mass of the body relative to the respective reference quantity, or reference position, a decision may be made relating to the movement that the user of the orthosis or prosthesis is currently carrying out. If, for example, the center of mass of the body during a flexion of the knee joint remains constantly behind the knee joint axis as well as behind the ankle joint axis, it is to be assumed that the user is sitting down or at least not carrying out a kneeling movement or wishes to kneel down. With the aid of this information, which is transmitted in addition to other items of information to the control device, a decision is then made relating to whether blocking or an increase of the resistance is induced or not induced, or another variation of the resistance is initiated.

Exemplary embodiments of the invention are explained in more detail below with the aid of the appended figures, in which:

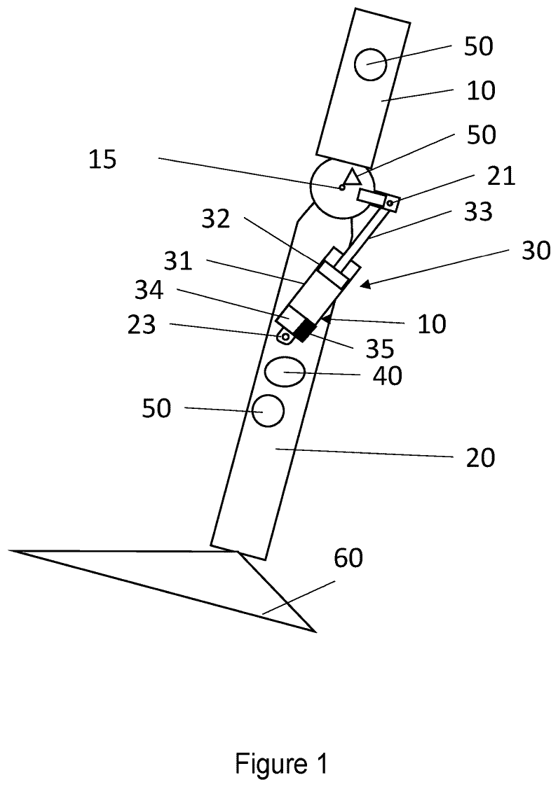

FIG. 1 shows a schematic representation of a prosthesis;

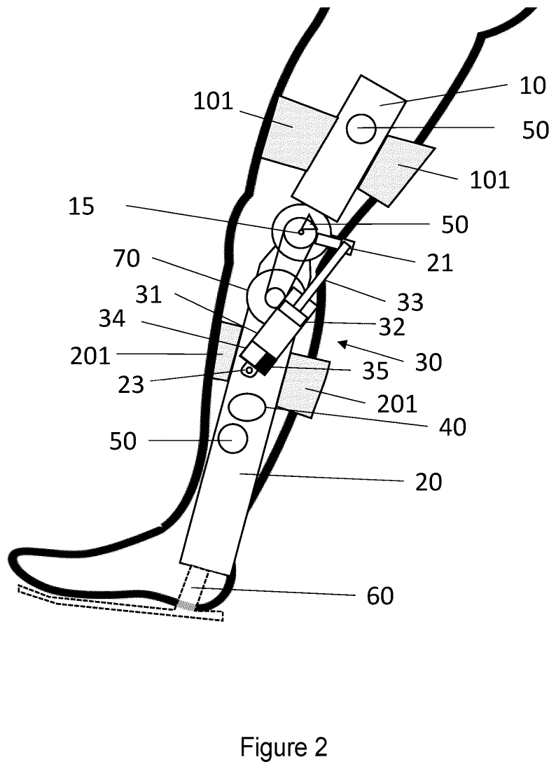

FIG. 2 shows a schematic representation of an orthosis;

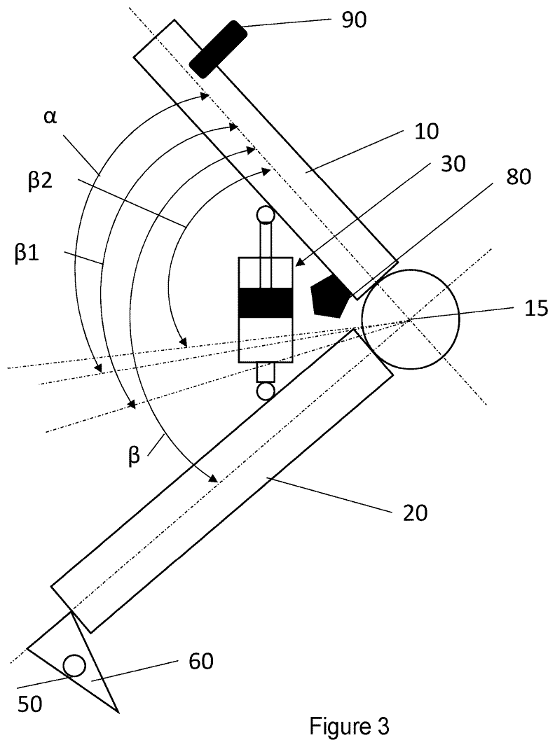

FIG. 3 shows a further diagrammatic representation in a flexed setting; and

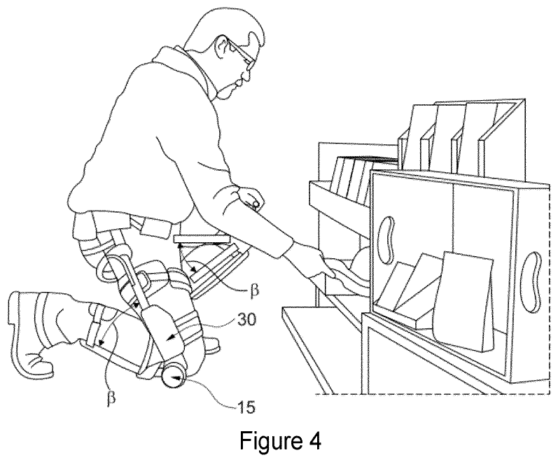

FIG. 4 shows an exemplary use.

FIG. 1 presents a schematic representation of a prosthetic knee joint as part of a prosthesis, and FIG. 2 an orthotic knee joint as part of an orthosis. The prosthetic knee joint comprises an upper part 10 and a lower part 20, which are mounted pivotably about a pivot axis 15 on one another. On the lower part 20, in an embodiment as a prosthesis, a prosthetic foot 60 is arranged on the distal end, and in the embodiment of the artificial knee joint as an orthotic knee joint, as shown in FIG. 2, the lower part 20 is configured as a tibial splint on which a foot part is not arranged, although an optional foot part 60 (which is represented by the dashed line) may be arranged on it. For the case of a KAFO, a foot part 60 on which a foot can be placed is arranged on the lower part 20. In order to produce a pure knee orthosis, however, this may also be omitted. In the embodiment as a prosthetic leg according to FIG. 1, a prosthesis socket or another device for receiving a femoral stump or for fixing to a person is arranged or formed on the upper part 10. In the embodiment according to FIG. 2, the orthosis is fixed to a leg by fastening means 101, 201, which are configured by way of example as straps, shells or the like, in order to fix the orthosis removably to the leg.

A resistance device 30 in the form of a linearly acting hydraulic damper is arranged between the upper part 10 and the lower part 20. In the exemplary embodiment represented, the hydraulic actuator 30 is configured with a hydraulic chamber or a cylinder, which is arranged or formed in a housing or base body 31. A piston 32 is mounted movably in the cylinder. The piston 32 is displaceable along the longitudinal extent of the cylinder and is fastened to a piston rod 33, which protrudes from the housing or base body 31. The piston 32 subdivides the cylinder into chambers, which are in fluidic communication with one another via a hydraulic line. The base body 31 or the housing may be mounted pivotably on the lower part 20 at a fastening point 23, in order to prevent twisting of the piston 32 during a pivoting movement of the upper part 10 relative to the lower part 20. The end of the piston rod 33 facing away from the piston 32 is fastened to the upper part 10, in the exemplary embodiment represented on a bracket in order to increase the distance from the pivot axis 15, at an upper fastening point 21. During a flexion, the piston 32 is pressed downward so that the volume of a flexion chamber is reduced and the volume of an extension chamber is correspondingly increased, less the volume of the piston rod 33 that is inserted. Arranged in the housing 31 in order to generate a pressure inside one of the chambers, there may be an electric motor 40 which drives in a pump (not represented) in order to apply a pressure to the hydraulic fluid inside one of the two chambers and thereby move the piston 32 in one direction or the other inside the cylinder. This brings about a flexion movement or an extension movement of the orthopedic device in the form of the prosthetic leg. The electric motor for driving the pump is an option which may be used in one embodiment in combination with the linear damper 30. In principle, a drive or motor is not necessary in a passive prosthetic knee joint. Instead of a linear damper, in particular linear hydraulics, an alternative embodiment of the resistance device provides a rotary damper, in particular rotary hydraulics, a magnetorheological resistance device or an electric motor, for example in generator mode. A combination of a plurality of the aforementioned resistance devices is also implemented in one embodiment.

Arranged inside the housing 31 or on the housing 31, there is an actuator 34 which is coupled to at least one control valve 35 by which the hydraulic resistance in the resistance device 30 can be varied. The actuator 34 is coupled to a control device 40, which activates, deactivates or modulates the actuator 34 on the basis of sensor values so as to be able to provide an adapted resistance and optionally hydraulic locking. In an embodiment of the resistance device 30 as a magnetorheological resistance device, the variation of the resistances takes place by activation, deactivation or modulation of a magnetic field, the actuator 34 then being the electromagnet or the solenoid.

At least one sensor 50 for recording the spatial orientation of the lower part 20, or of the upper part 10, is respectively arranged both on the upper part 10 and on the lower part 20. In particular, the sensor 50 is arranged only on the upper part 10 in order to record the spatial orientation. By this sensor 50, which may for example be configured as an IMU (inertial measurement unit), the spatial angle or the absolute angle with respect to a defined spatial orientation, for example the direction of gravity, is ascertained during the use of the prosthetic knee joint or orthotic knee joint. Instead of being configured as an IMU for recording spatial attitudes, the sensor 50 may also record other state data, in particular state data that relate to the artificial knee joint. Positions, angular settings, speeds, accelerations, forces as well as profiles or changes thereof, are in particular recorded as state data. The ascertained spatial angle of the upper part 10 and/or of the lower part 20, or another state variable, is compared with a threshold angle. When a threshold value, which is stored in a control 40 for the respective sensor value or a quantity derived therefrom, is reached or exceeded, the actuator 34 is modulated, activated or deactivated in order to vary the flow resistance in the resistance device 30, in the embodiment as a hydraulic damper, the viscosity, the braking force or the force opposing the flexion movement in another way.

The resistance device 30 in an artificial knee joint is conventionally used to modulate a flexion movement and an extension movement, in order to generate or assist a suitable or desired movement sequence. An extension movement is optionally assisted and advantageously braked shortly before a maximum extension is reached, in order to avoid a hard stop. A flexion movement is braked or suppressed in the stance phase and in the swing phase in order to ensure limitation of the flexion. In order to be able to drive the actuator 34, an energy store, particularly in the form of an accumulator, is likewise assigned to the actuator 34. The energy store may be arranged immediately next to the actuator 34 or alternatively elsewhere in the orthopedic device, where more space is available or this is deemed to be advantageous because of the weight distribution.

One difference between the embodiment according to FIG. 1 and the embodiment according to FIG. 2 is that a drive 70 is provided according to FIG. 2, in which an electric motor 70 is coupled to a pulley, optionally via gearing. By a V-belt or toothed belt, a flexion or extension of the knee joint may then be brought about or assisted, depending on the direction of rotation of the motor 70. The embodiment having the drive with an electric motor 70 via a mechanical force transmission device and parallel damping by a hydraulic damper may also be employed in a prosthetic knee joint. As already mentioned in connection with FIG. 1, the resistance device in an orthosis may also be formed by a motor, for example in generator mode.

The direct mechanical coupling of the electric motor as a resistance device 30 to the upper part 10 and the lower part 20 may take place via a force transmission device, for example via a spindle drive so that, instead of a piston rod 33, a spindle rod is inserted into or retracted from the housing 31 by the rotation of a spindle nut, which is driven by the electric motor. The motor as a resistance device is in another embodiment coupled via a gearing device to the upper part 10 and the lower part 20, for example via a planetary gear, in order to bring about, or brake, and influence a displacement of the upper part 10 relative to the lower part 20.

Furthermore, the control device 40 and at least one angle recording device as a sensor 50 are arranged on the prosthesis or orthosis. The angle recording device 50 records the angle between the upper part 10 and the lower part 20 and is configured, for example, as a direct angle sensor that detects the angle directly. Alternatively, the angle between the upper part 10 and the lower part 20 may be ascertained by evaluating the sensor data of two spatial attitude sensors 50. The two methods may also be used simultaneously or complementarily. All the sensors arranged on the prosthesis or orthosis are coupled to a control device 40, and their sensor values are used as the basis for controlling the actuator 34 of the resistance devices 30, when it is configured as a damper, or as input signals for a motor control when the resistance device 30 is configured as a motor. For the case of magnetorheological damping, the sensor values are used to control the magnetic field, or its variation. The actuator 34 is driven on the basis of the sensor data, in particular the spatial attitudes and/or angular positions, as well as position data and data relating to the loading, orientation, acceleration and/or deformation of further components, for example in order to activate, deactivate or modulate the electric motor 70, for example so as to reduce or increase a pivoting resistance, to limit an end-stop and/or to generate or assist a relative movement between the upper part 10 and the lower part 20.

FIG. 3 presents a schematic representation of a prosthetic knee joint or orthotic knee joint, having an upper part 10, a lower part 20 and a resistance device 30 between the upper part 10 and the lower part 20, in a flexed setting. The upper part 10 can pivot relative to the lower part 20 about the pivot axis 15 against the resistance of the resistance device 30. A joint angle β between the upper part 10 and the lower part 20 is in this case varied. In the exemplary embodiment represented, the joint angle β is measured on the rear side between the upper part 10 and the lower part 20. In a fully extended position, the joint angle β is 180°, and with increasing flexion the joint angle β decreases accordingly by the pivoting angle. The pivoting is mechanically limited by the mechanical end-stop 80, which is schematically represented and may for example also be formed by the piston rod of the resistance device 30 reaching the fully inserted position. When the maximum mechanical end-stop 80 is reached, the joint angle β2 is minimum. When the lower part 20 reaches previously defined positions before reaching the mechanical end-stop 80 with the joint angle 32, namely when the blocking angle α is reached, the resistance in the resistance device 30 is increased in order to suppress or more strongly damp a further flexion movement. The recording of the joint angle β takes place by one or more sensors, as described above. The sensor values are transmitted to the control device 40, which in one embodiment is adapted to increase the resistance in the resistance device 30 even before the blocking angle α is reached, namely after a threshold value β1 is reached, in order to prevent a hard stop in the mechanical end-stop 80. Alternatively or complementarily to an automatic increase of the flexion resistance when reaching either the blocking angle α or a threshold value β1 before the blocking angle α, the flexion resistance may take place because of a defined orientation of the upper part 10 in space or, for example, because of an unchanged joint angle β or another defined movement pattern, either of which is carried out over a particular period of time.

Furthermore, a blocking command may be transmitted to the control device 40 shown in FIGS. 1 and 2 via an input device 90, for example a switch on the upper part 10 or via another input device, for example a remote control or a microphone with speech recognition, in order either to increase the resistance before the blocking angle α is reached or to increase the resistance independently of whether the blocking angle α is reached, in particular to the extent of blocking the joint. likewise be provided with or coupled to sensors 50 that in turn are coupled to the control device 40 and record a load on the foot part 60 or pivoting or a position of the foot part 60 in the space or relative to the lower part 20. If, for example, a load of the forefoot part is detected in combination with a moment in the dorsiflexion direction, in the event of a simultaneous varied position of the joint angle β, a kneeling position may possibly be inferred so that automatic locking and blocking may take place by the resistance device 30. Alternatively, in a kneeling position, blocking of the artificial knee joint may be blocked by actuating the actuation device 90. The loads recorded in the setting, for example forces or moments about the pivot axis 15 and/or forces or moments on the foot part 60, or the mounting of the foot part 60 relative to the lower part, may be used to bring about release of the locking and enable a further flexion. By recording the corresponding sensor values and assigning the recorded values to particular situations or assumed situations, blocking of the flexion movement may be negated again once it has taken place. The flexion blocking may also be negated in the event that an extension is detected, in which case extension may advantageously always be possible.

FIG. 4 shows an artificial orthotic knee joint when being worn by a person, in which a resistance device 30 that blocks a flexion movement at the joint angle β represented is arranged on the upper part. The user may actively and deliberately induce the blocking by actuating an actuation element, which is not represented. Alternatively or complementarily, control is adapted so that the resistance device 30 automatically increases the flexion resistance before the mechanical end-stop is reached and blocks the flexion before the mechanical end-stop is reached, or at least damps it to such an extent that a hard stop of the lower part into the mechanical end-stop does not take place when the mechanical end-stop is reached. The automatic blocking may also take place for example when the user is in the kneeling position for a prolonged time, for example in order to pick up an object or perform activities in the kneeling setting. If the center of mass of the body then lies behind the pivot axis 15, the user can be supported on the orthosis with the resistance device 30 blocked. The same behavior is also achieved when using a prosthetic knee joint. It may be seen in FIG. 4 that the flexion blocking may be employed both for a lowered knee, in FIG. 4 the right knee, and for the raised, flexed knee, in FIG. 4 the left knee. Particularly by recording the position of the shank and therefore of the lower part, or the load on a foot part, it is possible to distinguish whether and which joint is being manipulated, i.e. the flexion resistance which is being increased and whether or not blocking is being employed.

The automatic activation and adaptation both in the orthosis and in the prosthesis takes place in such a way that hydraulic blocking and raising of the flexion resistance are brought about before the mechanical end-stop is reached, and may furthermore be calculated and adapted to the patient in question according to the requirements of the knee while taking into account the profile of the respective lever arm variation during flexion.

By a suitable selection of the threshold value β1, it is also possible to facilitate kneeling down. For example, if it is recognized by evaluating the sensors 50 that the patient wishes to kneel, the threshold value β1 may be defined comparatively early before the blocking angle α is reached, and a controlled genuflection movement may be induced after the threshold value β1 is reached. In particular, this takes place by deliberate triggering of the corresponding function, i.e. the actuation of a button, switch or for example voice input. The devices for this may be located on the prosthesis for orthosis, for example the prosthesis socket or an orthosis splint itself, or they may be arranged in a remote control. The remote control may for example be wirelessly coupled to the control device, for example in the form of an app on a smart device, a smartphone or the like. Personal configuration is also possible by using an app on the smart device, on which an orthosis or prosthesis control is stored. The speed with which the knee is then flexed, or can be flexed, may be adapted individually to the patient in question. A decision relating to whether the patient wishes to kneel down or wishes to carry out another activity, for example sitting down, may take place by ascertaining of the center of mass of the body relative to the treated leg. While the center of mass is displaced backward after a certain pivoting angle β is reached when sitting down, when kneeling down the center of mass remains in the region of the force transmission of the ground reaction force vector in the foot part, or in the direction in front. For example, a decision may therefore take place relating to whether the patient wishes to kneel down or sit down. The blocking angle α may also be adapted as a function of the detected situation, and also as a function of a triggering command by an actuation device. Conventionally, joint angles β of about 35° to 45° are reached when kneeling down, after which blocking is expediently possible. The blocking angle α may therefore also be set at a joint angle β of 45°, for example, when this is so desired by the patient. Shortly before the blocking angle α is reached, the flexion resistance is then increased and a further flexion is then optionally prevented after the blocking angle α is reached. Unlike when sitting down, the flexion resistance is not subsequently reduced in order to facilitate sitting after a maximum resistance is reached to assist the sitting movement, rather it is further increased or blocking is induced in order to dissipate the bodyweight or the loads via the orthosis or prosthesis.

1. A prosthetic or orthotic knee joint having an upper part and a lower part which is mounted pivotably about a pivot axis on the upper part and having a resistance device between the upper part and the lower part, with which the pivotability of the upper part relative to the lower part at least in flexion direction can be influenced, and having a control device which is coupled to the resistance device and by which the resistance against a flexion is varied as a function of the setting of the upper part with respect to the lower part, characterized in that the control device is adapted to block or more strongly damp a flexion movement at a blocking angle (α) when or before a mechanical end-stop is reached.

2. The prosthetic or orthotic knee joint as claimed in claim 1, characterized in that the control device is adapted to increase the flexion resistance over a defined angle range to the extent of blocking before the blocking angle (α) is reached.

3. The prosthetic or orthotic knee joint as claimed in claim 1, characterized in that the control device is coupled to at least one sensor for recording the joint angle (β) between the upper part and the lower part or the orientation of the upper part in space, and is adapted to vary the resistance due to the resistance device on the basis of the sensor values, in particular to increase the resistance to the extent of blocking after reaching a threshold value (β1) of the joint angle (β) and/or the duration (t1) of an unchanged joint angle (β) or an orientation of the upper part and/or a blocking command.

4. The prosthetic or orthotic knee joint as claimed in claim 1, characterized in that the control device is coupled to at least one sensor for recording a release signal, with which the blocking is negated.

5. The prosthetic or orthotic knee joint as claimed in claim 2, characterized in that the control device is adapted to increase the flexion resistance in the event of increasing flexion.

6. The prosthetic or orthotic knee joint as claimed in claim 1, characterized in that at least one control valve or an adjustment device is arranged in the resistance device and is coupled to an actuator that is activated, deactivated or modulated by the control device.

7. The prosthetic or orthotic knee joint as claimed in claim 1, characterized in that the resistance device is configured as a linear damper or rotary damper, in particular as hydraulics, pneumatics, a magnetorheological resistance device or drive.

8. The prosthetic or orthotic knee joint as claimed in claim 1, characterized in that parameters for the blocking can be set in the control device

9. The prosthetic or orthotic knee joint as claimed in claim characterized in that the blocking angle (α) is set at between 2° and 40°, in particular between 5° and 30° before the mechanical end-stop.

10. A method for controlling the prosthetic or orthotic knee joint as claimed in claim 1, characterized in that the flexion resistance is increased before the blocking angle (α) is reached, after reaching a threshold value (β1) of a joint angle (β), in the event of increasing flexion and/or in the event of a constant joint angle (β) or constant orientation of the upper part in space over a defined period of time and/or a blocking signal.

11. The method as claimed in claim 10, characterized in that the resistance is increased linearly or progressively.

12. The method as claimed in claim 10, characterized in that the flexion resistance is increased over an angle range of more than 5° before the blocking angle (α) is reached.

13. The method as claimed in claim 10, characterized in that the control device is coupled to a foot part and blocking and/or release takes place on the basis of the setting and/or load of the foot part.

14. The method as claimed in claim 10, characterized in that the position of the center of mass of the body relative to the treated leg is recorded or ascertained, and a decision relating to an increase in the resistance or blocking is made during a movement on the basis of the profile of the center of mass of the body.
